(19) United States
(12) Patent Application Publication
(10) Pub. No.: US 2021/0110910 A1
(43) Pub. Date: Apr. 15, 2021

(54) NUTRITION METHODS

(71) Applicant: ICON Health & Fitness, Inc., Logan, UT (US)

(72) Inventors: Megan Jane Ostler, North Logan, UT (US); Michelle Elaine Alley, Logan, UT (US)

(21) Appl. No.: 17/014,935

(22) Filed: Sep. 8, 2020

Related U.S. Application Data
(60) Provisional application No. 62/897,113, filed on Sep. 6, 2019.
(60) Provisional application No. 62/912,451, filed on Oct. 8, 2019.

(51) Int. Cl. G16H 20/60; G16H 10/20

(52) U.S. Cl. CPC G16H 20/60; G16H 10/20

(57) ABSTRACT

A nutrition plan includes a plurality of nutrition skills. Each nutrition skill is divided into several nutrition subskills. The nutrition skill is presented to the user. After an elapsed period of time, the user is asked a nutrition related question. The user responds, and a nutrition progress report is received. The nutrition progress report is checked against a nutrition goal. If user meets the goal, a second nutrition skill is presented. If the user does not meet the goal, then the first nutrition skill is repeated until the user meets the goal.

### CROSS-REFERENCE TO RELATED APPLICATIONS

This application claims priority to provisional patent application No. 62/897,113 entitled “NUTRITION METHODS” filed Sep. 6, 2019, and provisional patent application No. 62/912,451 entitled “NUTRITION METHODS” filed Oct. 8, 2019, which applications are herein incorporated by reference for all that they disclose.

### BACKGROUND

### Background and Relevant Art

Many people follow diets to improve their health and lose weight. Some diets focus on a specific aspect of health, such as protein, carbohydrates, fat, and so forth. Many diets may help a user to lose weight or improve health. The effectiveness of a diet may be dependent upon the user involvement. Some diets involve a large change in lifestyle. Some users may not be able to begin or maintain the diet due to the change in lifestyle. Some users may experience weight gain and lose the health benefits after stopping adhering to a diet.

### BRIEF SUMMARY

In some embodiments, a method for improving a user's health includes presenting a first nutrition skill to the user. The first nutrition skill includes a first nutrition action to be performed by the user. The user is asked a first nutrition-related question based on the first nutrition action after the first elapsed period of time. A first nutrition progress report is received based on a first response to the first nutrition-related question. The first nutrition progress report is checked against the first nutrition goal. If the first nutrition progress report meets or exceeds the first nutrition goal, a second nutrition is presented to the user. If the first nutrition progress report does not meet the first nutrition goal, the method is repeated until the first nutrition progress report meets or exceeds the first nutrition goal.

In other embodiments, a method for improving a user's health includes presenting a plurality of nutrition skills to a user. The plurality of nutrition skills includes eat more vegetables; eat protein throughout the day; stay hydrated; sweeten with fruit; power up with whole grains; eat healthy fats; fill the day with fiber; eat less sugar; honor hunger; increase variety; healthy dining out; and meal planning. Nutrition goals are set for each nutrition skill of the plurality of nutrition skills. The user is advanced successively through each nutrition skill of the plurality of nutrition skills based on whether the user meets or exceeds the nutrition goal for the previous nutrition skill.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of a nutrition computing system; according to at least one embodiment of the present disclosure;

FIG. 2 is a representation of a nutrition system, according to at least one embodiment of the present disclosure;

FIG. 3 is a representation of a method for improving a user's health, according to at least one embodiment of the present disclosure; and

FIG. 4 is a representation of a hunger/fullness chart, according to at least one embodiment of the present disclosure.

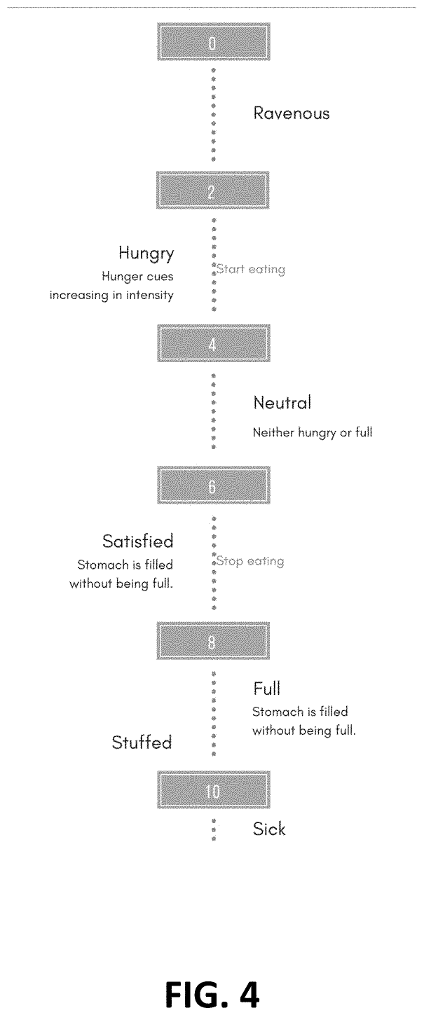

### DETAILED DESCRIPTION

This disclosure generally relates to systems and methods for improving a user's health. Specifically, this disclosure provides a series of nutrition skills or steps to a user, and following up with the user to determine the wellness of the user and to determine whether the user has complied with nutrition goals as part of the nutrition skill. In some embodiments, a single nutrition skill may be worked on at one time. In this manner, the user will develop healthy habits to change long-term behavior, which may lead to a healthier life. When a user meets a goal associated with the nutrition skill, the user may be advanced to the next nutrition skill in the list of nutrition skills. In some embodiments, a user may work on more than one nutrition skill at once.

The nutrition skills include a list of at least 12 skills, including: eat more vegetables; eat protein throughout the day; stay hydrated; sweeten with fruit; power up with whole grains; eat healthy fats; fill the day with fiber; eat less sugar; honor hunger; increase variety; healthy dining out; and meal planning. Each of these nutrition skills will help a user to develop healthy habits, which may lead to weight loss, a healthier metabolism, a greater sense of well-being, and other health improvements.

Each of the 12 nutrition skills includes one or more nutrition subskills. The broad nutrition skills include the general description of the healthy habit to be developed, and the nutrition subskills include the fine details of how to develop the healthy habit. In some embodiments, a single nutrition subskill will be worked on at once. When the user meets the nutrition goal associated with the single nutrition subskill, the user may advance to the next nutrition subskill. By gradually applying nutrition subskills, the user may not get overwhelmed by any single nutrition skill. Furthermore, by gradually applying nutrition skills and nutrition subskills, the user will develop healthy habits that may change the user's lifestyle. This may lead to permanent change in the user, thereby improving the user's life.

The user may be contacted and coached in a plurality of ways. For example, a nutrition coach, such as a nutritionist or a dietitian, may contact the user on a regular schedule, such as monthly, weekly, multiple times per week, daily, and multiple times per day. In some examples, the user may receive alerts on an electronic device, such as a phone, a tablet, a smartwatch, a computer, and so forth. The alert may include reminders to perform a nutrition action or assignment, encouragement for the user to reach his goals, educational information, and other information and alerts. In some embodiments, the alert may be automatically generated by an application. In some embodiments, the alert may be generated by the user. For example, the user may set a reminder in his phone to perform the nutrition action. In some embodiments, the alert may be provided by the nutrition coach in the form of a text message, an instant message through a nutrition or fitness application, a message through social media, or other message. In some embodiments, the user may be held contacted or coached using a combination of personal contact from the nutrition coach and alerts to a mobile device. In some embodiments the user may receive the alert at a specific time during the day, such as at a mealtime, snacktime, meal preparation time, or other time. In some embodiments, the alert may sound at a time before a nutrition event, such as mealtime, snacktime, meal preparation time, and so forth, such as 1 minute before, 5 minutes before, 15 minutes before, 20 minutes before, 30 minutes before, 1 hour before, and any time therebetween.

In some embodiments, the user may not interact with a nutrition coach, but may progress through the nutrition skills through interfacing with an application, such as an app for a computing device, a mobile device (e.g., smartphone, tablet, smartwatch), an exercise device operating system, other applications, and combinations of the foregoing.

The application may be controlled by a combination of hardware and stored memory that stores instructions to operate the application. The instructions may be executed by one or more processing resources. Processing resources include at least one processor and other resources used to process the programmed instructions. As described herein, the memory resources may represent generally any memory capable of storing data such as programmed instructions or data structures used by the adjusting system.

The processing resources may include I/O resources that are capable of being in communication with a remote device that stores user information, user preferences, nutrition goals, nutrition actions, reminders, nutrition skills, exercise information, exercise history information such as calories burned, diet information, other types of information, or combinations thereof. The remote device may be a mobile device, a cloud based device, a computing device, another type of device, or combinations thereof.

The remote device may execute a program that can collect and/or display nutrition information with respect to the user. An example of a program that may be compatible with the principles described herein includes the iFit program which is available through www.ifit.com. An example of a program that may be compatible with the principles described in this disclosure is described in U.S. Pat. No. 7,980,996 issued to Paul Hickman. U.S. Pat. No. 7,980,996 is herein incorporated by reference for all that it discloses. In some examples, the user information accessible through the remote device includes the user's age, gender, body composition, height, weight, health conditions, other types of information, or combinations thereof.

The processing resources, memory resources, and remote devices may communicate over any appropriate network and/or protocol through the input/output resources. In some examples, the input/output resources include a transmitter, a receiver, a transceiver, or another communication device for wired and/or wireless communications. For example, these devices may be capable of communicating using the ZigBee protocol, Z-Wave protocol, BlueTooth protocol, Wi-Fi protocol, Global System for Mobile Communications (GSM) standard, another standard, or combinations thereof. In other examples, the user can directly input some information into the nutrition system through a digital input/output mechanism, a mechanical input/output mechanism, another type of mechanism, or combinations thereof.

The memory resources may include a computer readable storage medium that contains computer readable program code to cause tasks to be executed by the processing resources. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory, or combinations thereof.

A nutrition application may include nutrition information such as nutrition skills, nutrition subskills, nutrition goals, nutrition actions, and so forth. The nutrition application may further include user input, where the user may input compliance with nutrition actions, food eaten, water consumed, and so forth. The application may provide reminders to the user. In some embodiments, the user may follow the nutrition program exclusively on the nutrition application. In some embodiments, the user may interact with a nutrition coach. In some embodiments, the user may interact with both the application and a nutrition coach.

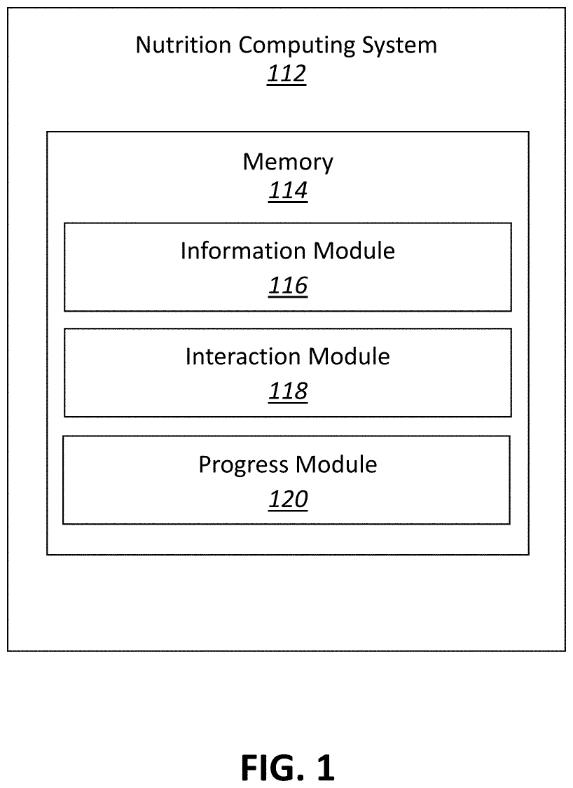

FIG. 1 is a representation of a nutrition computing system 112, according to at least one embodiment of the present disclosure. The nutrition computing system 112 may include the nutrition application used to interact with the user. The nutrition computing system includes a memory 114 that includes instructions, which, when accessed by the nutrition computing system 112, cause the nutrition computing system 112 to perform commands. The memory 114 includes an information module 116. The information module 116 may include information about the nutrition program, such as information about the nutrition skills and subskills, background information regarding the nutrition program, and tips and tricks for compliance with the nutrition program.

The memory 114 includes an interaction module 118. The interaction module 118 may allow a user answer one or more questions regarding nutrition history, compliance with nutrition goals, existing knowledge of nutrition skills, and others. The interaction module 118 may further allow the user to interact with a nutrition coach. For example, the interaction module 118 may include video chatting modules, picture modules, text messaging, instant messaging, emailing, and other modes of interaction. The interaction module 118 may allow the user to transmit messages to the nutrition coach, and/or the nutrition coach to transmit messages to the user. In some embodiments, the nutrition coach may access information provided by the user in the interaction module to tailor the nutrition plan to the user.

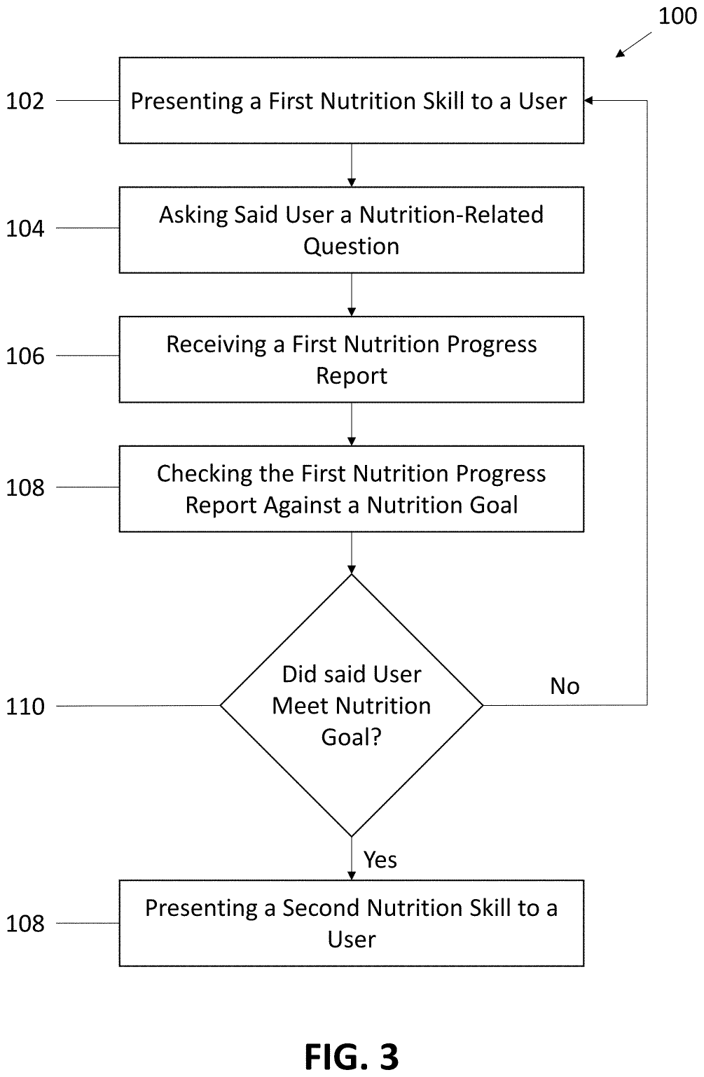

The memory 114 includes a progress module 120. The progress module 120 includes one or more algorithms, such as the algorithm disclosed in FIG. 3, that are used to determine the progress of the user with the nutrition plan. For example, the progress module 120 may include instructions to analyze the responses provided by the user at the interaction module 118. In some embodiments, the progress module 120 may analyze this information automatically. In some embodiments, the progress module 120 may be monitored by a nutrition coach. The nutrition coach may analyze the responses by the user at the interaction module 118. In some embodiments, the nutrition coach and instructions in the progress module 120 may cooperate to analyze the nutrition progress of the user.

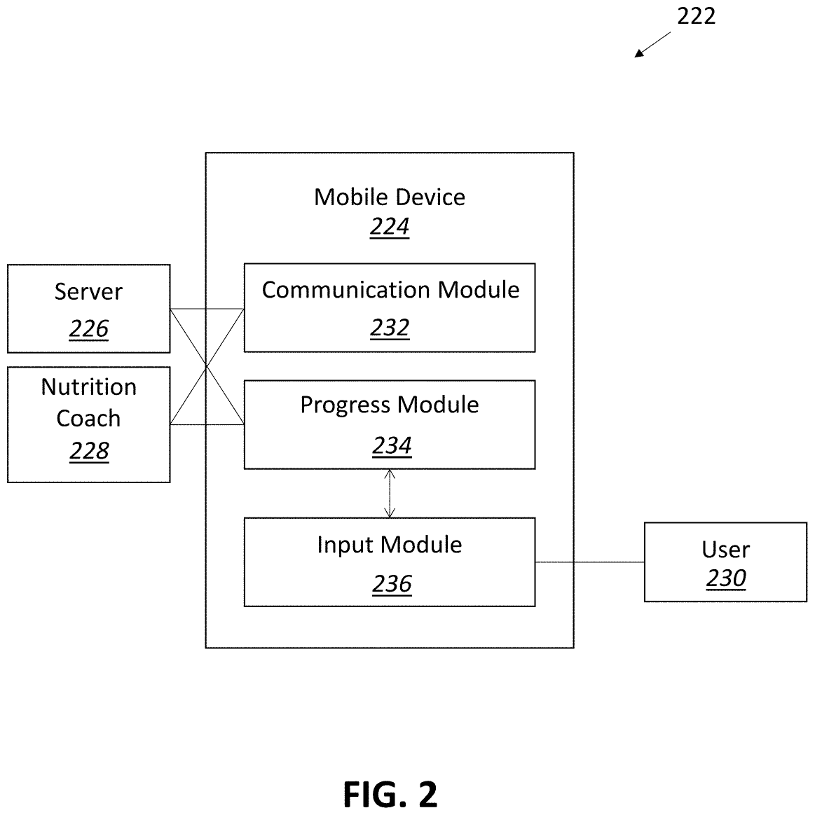

FIG. 2 is a representation of a nutrition system 222, according to at least one embodiment of the present disclosure. The nutrition system 222 includes a mobile device 224, a sever 226, a nutrition coach 228, and a user 230. The server 226, the nutrition coach 228, and the user 230 may all be in communication the mobile device 224. The mobile device 224 may include a plurality of modules, which include instructions, which when accessed by one or more processors on the mobile device 224, may cause the processors to perform functions. The modules may include a communication module 232, a progress module 234, and an input module 236.

The communication module 232 may provide communication between the mobile device 224 and the server 226 and/or the nutrition coach 228. For example, the server 226 may include details about the nutrition skills and/or nutrition subskills. When a user is ready to work on a specific nutrition skill or nutrition subskill, the mobile device 224 may request nutrition information from the server 226, and the server 226 may communicate the nutrition information to the mobile device 224 using the communication module.

In some embodiments, the nutrition coach 228 may communicate with the mobile device 224 through the communication module 232. The nutrition coach 228 may provide instruction, encouragement, information, reminders, and other information to the user 230 through the communication module 232. The progress module 234 may track the progress of the user 230. The progress module 234 may communicate with the server 226 to receive information regarding questions to ask the user specific to nutrition skills and nutrition subskills. The progress module 234 may communicate with the nutrition coach 228. The nutrition coach 228 may receive information from the progress module 234 regarding the user's nutrition progress. The nutrition coach 228 may further provide the progress module 234 with questions to ask the user 230 to help determine the user's 230 nutrition progress.

The user 230 may input nutrition information into the nutrition system 222 in the input module 236. For example, the user 230 may input information regarding the user's height, weight, age, sex, nutrition history, eating habits, other nutrition information, and combinations thereof. In some embodiments, the input module 236 may communicate the user information to the progress module 234, which may then communicate the information to the server 226 and/or the nutrition coach 228. In this manner, the user may interact with the nutrition system 222 through the mobile device 224.

In some embodiments, before advancing from one nutrition skill or subskill to another nutrition skill or subskill, the user may be asked one or more performance-related question. For example, the user may be asked if she ate the specified amount of the specified food. In other examples, the user may be asked if she at the specified food at the specified time. Until the user performs the specified nutrition action, the user will not advance to the next nutrition skill or subskill. By following up with the user and holding the user accountable, the user may better perform the nutrition actions.

FIG. 3 is a representation of an embodiment of a method 100 for improving a user's health. The method 100 includes presenting a first nutrition skill to a user at 102. The first nutrition skill may be associated or include a first nutrition action that is to be performed by a user. The first nutrition action may include any nutrition action, such as eating a specified food or group of food, eating an amount of the specified food or group of food, limiting intake of a specified food or group of foods, eating a specified food at specific times during the day, and so forth.

After a first elapsed period of time, such as a week, between two and six days, a day, or less than a day, the user may be asked a nutrition-related question based on her performance of the nutrition action at 104. For example, the user may be asked if she ate the specified food at the specified time. In other examples, the user may be asked if she ate the specified amount of the specified food. In some embodiments, the user may be asked the nutrition-related question by a nutrition coach. In some embodiments, the user may be asked the nutrition-related question by an application. In some embodiments, the user may volunteer information to the nutrition coach and/or the application before being asked the nutrition-related question.

In some embodiments, the nutrition-related question may include a plurality of questions. For example, the nutrition-related question may include a simple compliance question, such as “Did you perform the specified nutrition act?” In some examples, the nutrition-related question may include a wellness question, such as “How did performing the nutrition action make you feel this week?” “What were some challenges to accomplishing the nutrition action?” “What did you learn this week?” “What contributed to your success this week?” In some examples, the nutrition-related question may include multiple wellness questions, and/or a combination of compliance and wellness questions.

The application or the nutrition coach may then receive a first nutrition progress report based on a first response to the first nutrition-related question at 106. In some embodiments, the first nutrition progress report may include a mark by the user indicating whether or not he performed the first nutrition action. In some embodiments, the first nutrition progress report may be developed by the application and/or the nutrition coach based on the user's response to the nutrition-related question or questions.

The first nutrition progress report may then be checked against a nutrition goal at 108. In some embodiments, the nutrition goal may include performing the nutrition action. In some embodiments, the nutrition goal may be a wellness goal, such as whether or not the user felt an improvement to his health as a result of performing or attempting to perform the nutrition action.

When checking the first nutrition report against the first nutrition goal, a query of whether the user met the nutrition goal may be asked at 110. If the user did not meet the first nutrition goal, then the first nutrition skill may be presented to the user again, and the first nutrition skill process shown at acts 102 through 108 may be repeated until the user meets the first nutrition goal. In other words, the user may have multiple opportunities or tries to meet the first nutrition goal before advancing to the second nutrition skill. This may ensure that the user has developed a healthy habit with regards to the first nutrition skill before adding a new skill. In this manner, the user may focus on a single nutrition skill. This may help the user from feeling overwhelmed, which may result in the user giving up and no longer working on performing the nutrition action.

If the first nutrition progress report indicates that the user met the first nutrition goal, then a second nutrition skill may be presented to the user at 112. The method 100 may then be repeated with the second nutrition skill. Under this framework, the user may work on a single nutrition skill at a time. This may be a manageable way for the user to develop healthy habits and improve his or her health.

The nutrition skills include specific instruction to improve a user's health. In some embodiments, a user may progress through the nutrition skills in the following order: (1) eat more vegetables; (2) eat protein throughout the day; (3) stay hydrated; (4) sweeten with fruit; (5) power up with whole grains; (6) eat healthy fats; (7) fill the day with fiber; (8) eat less sugar; (9) honor hunger; (10) increase variety; (11) healthy dining out; and (12) meal planning. In some embodiments, the user may progress through the nutrition skills from a different starting point (e.g., starting with number (5) power up with whole grains). In some embodiments, the user may progress through the nutrition skills in a different order.

In some embodiments, a user may work on more than one nutrition skill at one time. For example, if a user feels like she has already has a specific nutrition skill, then the user may either skip that nutrition skill, or work on that nutrition skill simultaneously with another nutrition skill. In other examples, the user may work on skills (10), (11), and (12) while working on the remainder of the skills because those skills are applicable to skills (1) through (9). In this manner, the user may tailor the nutrition plan to match her needs, skills, and interests. This may help the user to develop healthy habits more quickly than working on a single nutrition skill at a time.

### Nutrition Skill 1: Eat More Vegetables

Vegetables include many vitamins, dietary fiber, minerals, important phytochemicals, and essential antioxidants that help prevent diseases. People who have a diet rich in vegetables may have a reduced risk for stroke, certain cancers, cardiovascular diseases, and other chronic diseases. Vegetables are also naturally low in calories and rich in nutrients. This means that, per calorie, vegetables are very nutritious.

While all vegetables include many nutrients, different vegetables include different nutrients. Therefore, eating a variety of vegetables will provide a user with a valuable variety of nutrients. For example, the different colors of different vegetables may provide an indication that the vegetables include different nutrients. Carrots, for example, contain the nutrients that make them orange, and they are different than the nutrients that make broccoli green. A variety of vegetables can help a user to satisfy her nutrient needs. Fortunately, there are many different types of vegetables, and therefore it may be possible for a user to acquire all the nutrients she needs. By the end of the nutrition skill of eat more vegetables, a user should be eating six servings of vegetables per day, distributed over breakfast, lunch, dinner, and snacks.

The nutrition skill of eat more vegetables may include the subskills of incorporating two servings of vegetables at lunch, incorporating two servings of vegetables at dinner, adding a vegetable to breakfast, and enjoying five cups of dark, leafy greens a week. The nutrition subskill of incorporating two servings of vegetables at lunch may include keeping a rough count of and measuring consumption of vegetables. For raw vegetables (like carrots), one cup is a serving. For leafy greens, a serving may be approximately two cups. For cooked vegetables, a serving may include approximately half of a cup. For juice, approximately 4 ounces of 100% juice may be a serving, although juice should be used sparingly as a serving. Using measuring cups to count vegetables throughout the day may be tedious; a user's hands may be used as a rough serving estimate. Although different users may have hands that are a different size, the user's hands may be a quick estimate of volume. The average fist is equal to about one cup and a handful is equal to about a half cup.

The nutrition subskill of incorporating at least two servings of vegetables at dinner may include filling half of a user's dinner plate with vegetables. Alternatively or additionally, vegetables may be incorporated into the main dish of a meal. In general, a dinner should include twice as many vegetables as protein or carbohydrates. For example, if a recipe does not call for twice as many vegetables as protein or carbohydrates, a user may simply double the vegetables or continue to add vegetables until this ratio is reached.

The nutrition subskill of incorporating a vegetable at breakfast may include preparing quick breakfasts that incorporate vegetables, such as a leftover vegetable frittata, a spinach smoothie, or an egg sandwich with tomatoes and sprouts. A vegetable rich breakfast may increase the amount of fiber in the breakfast, which may keep the user satisfied for longer during the day.

The nutrition subskill of incorporating dark, leafy greens into meals may include adding an extra vegetable serving to a meal or snack. Furthermore, this subskill may include adding dark leafy greens, including romaine lettuce, spinach, kale, swiss chard, collards, and arugula. These greens may have high concentrations of iron, potassium, vitamin A, vitamin C, and antioxidants. Incorporating leafy greens into a user's diet can help with digestion, promote healthy skin, maintain eyesight, and improve immune functions. Leafy greens may be used as a base for a salad, a topping, or an add-in to an existing meal. Leafy greens may add to the crunchy texture of a meal or may be cooked down and added to almost anything. A user should set a goal to eat dark, leafy greens most days of the week.

### Nutrition Skill 2: Protein Throughout the Day

Eating protein may help to build and maintain muscle mass, assist a user's appetite control, and provide other health benefits. Protein may be considered a building block for the body. For example, protein is used in the body's muscles, tissue, bones, hormones, and enzymes.

The timing of eating protein is critical to its effectiveness in being processed by the human body. It is important for omnivores to spread protein consumption throughout the day. Often, a user may consume a predominantly carb-based breakfast and lunch, then a large protein-heavy dinner, such as a 12-ounce steak, baked potato, broccoli, and a glass of milk for dinner. This dinner may include up to 94 grams of protein. While this may be an adequate amount of protein, protein consumed all at once like this may be less beneficial for the user. Thus, a user should consider eating protein throughout the day to receive its full benefit.

Protein may require more energy and takes longer to digest than carbohydrates and/or fats. This means that more protein may help a user with appetite control, regulate blood sugar levels (as it slows down carb digestion and absorption), and may increase the metabolism. Protein also helps build a user's muscles and protects against muscle breakdown when combined with strength training.

However, eating more protein is not always better. Excessive protein may be digested and utilized for energy, just like carbs and fat. That means that a user needs enough to reach your goals, but not so much that it ends up just being extra energy and calories. Thus, eating protein throughout the day may help to improve a user's health.

The nutrition skill of eat protein throughout the day may include the subskills of eating a high-protein breakfast, incorporating protein into snacks, limiting the intake of red and processed meats, and incorporating plant-based proteins into a user's diet. The nutrition subskill of eating a high-protein breakfast may help a user to start his day with adequate protein. This may help to refuel the user's muscles after the overnight fast. This may also help curb the user's hunger, thereby directing the user away from sugar-based breakfast foods. An adequate protein goal may include eating at least 20 grams of protein at breakfast.

The nutrition subskill of incorporating protein into snacks may help a user to view snacks as mini meals. Thus, snacks should be balanced like meals, and not be overly sugary. Good snacks may include fiber-rich carbohydrates and protein. This may help the user to eat more nutrient-rich foods, and the added fiber and protein may help a user to feel more satiated. In some embodiments, a user may add healthy fats (e.g., nuts) to the protein-based snack.

The nutrition subskill of limiting intake of red and processed meats may help to decrease a user's risk of heart disease and some cancers. Red and processed meats may be replaced with lean cuts of meat, fish, and plant-based proteins. While red meat may still be consumed in a healthy diet, a user may set a goal to limit red meat to no more than three times per week, and to choose lean, grass-fed cuts of meat where possible. Furthermore, processed meats like bacon and sausage may be limited to no more than one time per week. Processed meats may be replaced with meats like chicken, fish, or plant-based alternatives.

The nutrition subskill of incorporating plant-based proteins into a user's diet may include replacing at least a portion of meat-based proteins with plant-based proteins. A user may not need to become vegetarian or vegan, however, replacing meat-based proteins with plant-based proteins may benefit a user's health and the environment. For example, it is estimated that one pound of protein from kidney beans requires 18 times less land, 10 times less water, 9 times less fuel, 12 times less fertilizer, and 10 times less pesticide than producing one pound of beef. Furthermore, most nutrients found in meat may be found in plants. Plants also may include phytochemicals, beneficial fibers, vitamins, and minerals. Thus, while a healthy and balanced diet may include meat-based proteins, it may make sense for a user to consider replacing some meat-based proteins with plant-based proteins.

### Nutrition Skill 3: Stay Hydrated

People often meticulously count grams of protein or fiber, but they may forget about the importance of water. Water makes up more than two-thirds of the human body. Your hydration can affect both a user's athletic performance and journey to better health. Hydration aids in heat dissipation, metabolism, digestion, joint lubrication, energy, other bodily functions, and combinations of the foregoing. Poor hydration can lead to headaches, muscle cramps, and a reduction in blood volume.

Hydration can improve many things for a user's health. People who are well hydrated may lose more weight than their dehydrated counterparts. Since dehydration can lead to muscle fatigue, those who stay well hydrated also have more energy. Plus, they are better able to regulate their thermal temperatures, which allows them to push themselves harder during workouts.

To prevent this, a user may drink water throughout the day, and may drink at least eight ounces right before exercise. After exercise, a user may set a goal to replace lost fluids within two hours. For example, a user should drink 20-24 fluid ounces of water or a sports beverage for every pound lost during exercise. Losing too much weight to water during exercise may decrease a user's performance. For example, losing 1-2% of water weight during a workout can start to have negative impacts on the user's performance. Greater than 3% of water weight loss during a workout may lead to heat stroke of the user.

A user may have heard a conventional estimate of how much water to drink as being about eight glasses of water a day. While this rule of thumb may be adequate for some people, different people may require different amounts of water during the day. A user may consider using the following equation to estimate water consumption during the day.

(body weight (kg)−20)×15+1,500=fluid requirement (mL)

While this formula may provide a user with a baseline, a user may modify this amount based on exercise, diet, environment, other conditions, and combinations of the foregoing. Thus, rather than meticulously tracking the volume of water consumed, a user may use other cues to approximately determine a level of hydration. For example, a user may observe his urine color. Pale yellow, like a glass of lemonade, is an indication of proper hydration. In some examples, odorous urine may be a sign of dehydration.

The nutrition skill of stay hydrated may include the nutrition subskills of drinking a glass of water first thing in the morning, drinking a glass of water before each meal, paying attention to urine color, and limiting sweetened beverages. The nutrition subskill of drinking a glass of water may help a user set a routine of hydrating first thing in the morning. This may help a user to feel more energized during the day.

The nutrition subskill of drinking a glass of water before each may help a user to have at least three reminders each day to drink a glass of water. Furthermore, drinking a glass of water with each meal may help a user to eat less and avoid overeating. Still further, drinking water with meals may help a user to avoid fiber-related constipation.

The subskill of paying attention to urine color may include aiming for urine to be clear or pale yellow. Urine color may be a cue to indicate how hydrated a user is. The darker the color of the urine, the less hydrated the user. Furthermore, if a user's urine has an odor, then the user may be dehydrated. Thus, by paying attention to the cues provided in the user's urine, the user may be able to stay on top of his hydration.

The subskill of limiting sweetened beverages may include any beverage that includes added sugar, including soda pop, sports drinks, coffee, iced teas, juices, and so forth. Unsweetened tea, coffee, and so forth are not sweetened, and may still be consumed under this sub skill. A user may set a goal of less than 24 ounces of sweetened beverages per week. This may help a user to improve her health and reduce the amount of calories consumed.

Nutrition Skill 4: Sweeten with Fruit

Since fruit has natural sugar content, many nutrition programs encourage a user to limit fruit consumption. However, the sugar from fruit may be digested very differently than sugar from baked goods. That may be because fruit may include fiber as well as sugar. Fiber may slow down digestion and keep the fruit's natural sugar from spiking our blood sugar levels. Fruit also include vitamins, minerals, and phytochemicals. Phytochemicals are the compounds that give fruits their unique tastes, smells, and colors, and they contain important nutrients for our body. For example, many antioxidants are phytochemicals, which help with disease prevention.

Since fruit is sweet, it makes a great dessert or snack to satisfy sweet cravings. A user may set a goal to eat fruit between two and three times a day. Because fruit typically has more calories and sugar than vegetables, the goal for fruit consumption is less than that for vegetables. A serving of fruit may include ¼ cup of dried fruit, ½ cup of chopped fruit, 1 small whole fruit, ½ large fruit, or 4 ounces 100% juice. While 100% juice is a serving, juice does not include the fiber from the fruit, and therefore is a concentrated source of sugar and calories.

The nutrition skill of sweeten with fruit includes the nutrition subskills of incorporating fruit at breakfast and snacking on fruit. Some users may already eat fruit with breakfast, either as whole or chopped fruit or fruit juice. A user may set a goal to consume less fruit via fruit juice and consume more whole or chopped fruit at breakfast for the nutrition subskill of incorporating fruit at breakfast. This may help to provide the user with nutrients, and help fill the user with fiber, curb the user's appetite, and keep the user's blood sugar steady.

The nutrition subskill of snacking on fruit may include making fruit more available as a snack. For example, a user may have fruit already cut up in the fridge, may purchase more fruit at the store, or pack fruit as a snack at work. Fruits may be placed at eye level in the fridge, thereby placing it at eye level so it is the first thing seen when opening the fridge. Furthermore, fruit may be used to satisfy sweet cravings. Since fruit is already sweet, it may satisfy a user's sweet cravings without being too sugary or bad for the user.

Nutrition Skill 5: Power up with Whole Grains

The term whole grain means that all parts of the original grain are still intact, including the germ, endosperm, and bran. In refined grains, like all-purpose flour, the germ and bran have been removed, leaving just the starchy endosperm. This is important because the germ and bran contain many of the nutrients provided by the grain, such as B vitamins, fiber, and minerals (like iron).

When manufacturers take these vitamins, fiber, and minerals away, the remaining product may be high in carbohydrates without much nutritional value. That's why manufacturers enrich the refined products by adding back in some of the missing nutrients like B vitamins, but often don't add the fiber. Fiber is a carbohydrate that is indigestible by humans and is found mostly in whole grains, legumes, vegetables, and fruit. Fiber has a few important roles in the body, including lowering cholesterol, preventing inflammation, stabilizing blood sugar levels, avoiding insulin resistance, feeding the good bacteria in your gut, and preventing constipation. By eating whole grains instead of refined grains, a user may provide her body with important nutrients from carbohydrates, vitamins, minerals, fiber, and phytochemicals.

The nutrition skill of power up with whole grains includes the nutrition subskills of selecting whole-grain options at grocery stores and restaurants, trying new whole grains, and making at least half of grain products whole grain. The nutrition subskill of selecting whole-grain options at grocery stores and restaurants may include looking for the word “whole” in the ingredient list. Furthermore, a user may have an indication of whether a product is whole grain based on the fiber content; whole grains may include at least three grams per servings. Other ways to choose whole grains is to purchase grains in the whole form, such as barley, brown rice, oats, millet, or black rice. A user may further ask a waiter if there is a whole-grain option at a restaurant.

The nutrition subskill of trying new whole grains may add variety to the user's food consumption. A user may set a goal to try at least one new grain during the elapsed time period. Different whole grains include: amaranth, barley, buckwheat, bulgur, farro, freekeh, millet, quinoa, rye, spelt, and wild rice.

The nutrition subskill of making at least half of grain products whole grains may include eating whole grains consistently. By focusing on consistently ordering whole grain options and cooking with whole grains, the user may form the habit of consistently eating whole grains.

### Nutrition Skill 6: Eat Healthy Fats

Many users have an aversion to fat in their diet. Low-fat and nonfat products fill the shelves at local grocery stores. People in the past took the old saying, “You are what you eat.” to heart and thought if a person ate fat, he would become fat. That was one of the biggest lies that the American public has come to believe. People ran away from fat and made sure every product had a low-fat or nonfat option. However, fat may be healthy.

Fat is a macronutrient that is necessary for the human body to thrive and is therefore a necessary part of a healthy diet. The right kinds of fat are important for a user's brain, heart, vitamin absorption, insulin sensitivity, and even weight control. While there are many types, dietary fat is divided into two categories: unsaturated and saturated fats. Those categories are then further divided into different types, such as monounsaturated and polyunsaturated fats (which includes omega-3 fatty acids).

One of the reasons fat has been criticized is that it has more than double the calories per gram, compared to carbs and protein, while being more satiating to eat. Eating a food, meal, or snack that contains fat will satiate a user more and help you eat less, help control your appetite later on, and stabilize your blood sugars, preventing sugar highs and lows.

Monounsaturated fats are found in nuts, nut butters, avocados, and olives. Polyunsaturated fats are mostly found in plant-based products and oils. A good rule of thumb is that heart-healthy oils typically turn to liquid at room temperature.

An important group of polyunsaturated fats that the American diet often lacks are omega-3 fatty acids. These can be found in salmon, flax seeds, oils, walnuts, and sunflower seeds.

The two dietary fats that may be helpful to consume in moderation are saturated fat and trans fats. Saturated fats are typically solid at room temperature, like butter, lard, coconut oil, and the fat on meat. Trans fats can be found naturally in small amounts in food, but typically are found in commercially produced food products, such as baked goods, fried foods, and margarine.

Saturated fats increase LDL (e.g., bad cholesterol) and trans fats increase LDL and decrease HDL (e.g., good cholesterol), which may increase your risk for heart disease. Avoid trans fats that are found in commercial products. Be sure to check your labels for partially hydrogenated oils. For a healthy diet, it is as much about the amount of fat as it is the source of the fat. A user may set a goal to consume the majority of her fats from heart-healthy sources like fish, nuts, seeds, olive oil, avocados, and canola oil.

The nutrition skill of eating healthy fats includes the nutrition subskills of enjoying fatty, cold-water fish twice a week, consuming one ounce of nuts or seeds a day, and cooking with healthy fats. The nutrition subskill of enjoying fatty cold-water fish twice a week may provide a user with a good source of protein, vitamin D, and omega-3 fatty acids. Omega-3 fatty acids are a healthy form of fat that protect the heart by decreasing triglycerides, plaque build-up, and blood pressure. They help decrease inflammation and are an important part of a healthy diet. The American Heart Association recommends eating fish at least two times per week at 3.5 ounces per serving. Some fatty fish options include: salmon, mackerel, herring, lake trout, sardines, and albacore tuna. Avoid eating fish with high levels of mercury such as shark, swordfish, king mackerel, and tilefish. Fish may be supplemented with other omega-3 fatty acids.

The nutrition subskill of consuming one ounce of nuts or seeds daily may be performed by eating whole or chopped nuts, nut butters, nut pastes, oils, flours, milks, and combinations of the foregoing. While nuts and seeds are high in calories and fat, these monounsaturated and polyunsaturated fats (including omega-3s) help lower bad cholesterol and have been associated with a lower risk of heart disease. These heart-protecting fats in nuts are also accompanied by vitamins, minerals, fiber, and phytochemicals, such as antioxidants. However, those with nut allergies should of course not consume nuts. An ounce of nuts may include 47 pistachios, 24 almonds, 28 peanuts, 21 hazelnuts, 20 pecan halves, 18 cashews, 14 walnut halves, 10-12 macadamias, and 6-8 brazil nuts.

The nutrition subskill of cooking with healthy fats may include cooking with oils that are typically liquid at room temperature. These fats may include avocado, canola, and olive oil. This may help to decrease LDL cholesterol and increase HDL cholesterol.

Nutrition Skill 7: Fill the Day with Fiber

Many Americans do not eat enough fiber. The recommended amount of fiber is 25 grams for women and 38 grams for men. That's hard to get with the typical Western diet of meat, dairy, refined grains, sweet snacks, sugary beverages, and other processed foods that have little-to-no fiber. Fiber is a carbohydrate that is indigestible by humans and is found mostly in whole grains, nuts, seeds, legumes, vegetables, and fruit.

There are generally two types of fiber: soluble (e.g., dissolves in water) and insoluble (e.g., doesn't dissolve in water). When soluble dissolves in water, it creates a gel that cleans as it moves through our gastrointestinal tract. This is the fiber that helps lower cholesterol, slows digestion, and may have a beneficial effect on blood sugar levels. Soluble fibers are fermentable and help encourage the growth of healthy gut bacteria. Excellent sources of soluble fiber include legumes, oats, barley, nuts, flaxseeds, fruits, and vegetables.

Insoluble fiber is best known for its role in keeping people regular, as it increases movement through the GI tract. It helps with constipation, overall GI health, and may help with the prevention of GI disorders, such as diverticulosis and colon cancer. Some insoluble fibers are also fermentable and help encourage the growth of healthy gut bacteria. Insoluble fibers may be found in whole grains, wheat bran, nuts, seeds, fruits, and vegetables.

Fiber may be found in the food that a user should be eating as part of a healthy diet. Fruit and vegetables, legumes, nuts, seeds, and whole grains are great sources of fiber. However, the body needs to get used to fiber, or the user may have some gastrointestinal distress. It may be useful to slowly adjusting a user's diet to get more fiber.

The nutrition skill of fill the day with fiber includes the nutrition subskills of eating between five and ten grams of fiber at breakfast, incorporating fiber-rich foods as snacks, and incorporating at least two sources of fiber during every meal. The nutrition subskill of eating five to ten grams of fiber at breakfast should already be met with the previously achieved goals. This is because there is fiber in fruit (e.g., nutrition skill 4), vegetables (e.g., nutrition skill 1), whole grains (e.g., nutrition skill 5), nuts (e.g., nutrition skill 6), and seeds.

The nutrition subskill of incorporating fiber-rich foods as snacks may include pairing fiber-rich carbohydrates with protein for snacks. For example, snack combinations may include carrots with string cheese, apple and peanut butter, Greek yogurt and berries, whole-grain crackers or veggies with hummus, orange with cashews, cucumbers with tuna salad, and combinations of the foregoing.

The nutrition subskill of incorporating at least two sources of fiber during every meal may help a user to continue to focus on previous nutrition skills and goals. This is because fiber is found in many of the foods discussed in previous nutrition skills. A goal of two sources of fiber at every meal may help to reinforce the habits developed in previous nutrition skills.

### Nutrition Skill 8: Eat Less Sugar

The saying, “the dose makes the poison” may help to describe sugar consumption. At really high levels, most things are bad for the body, and that appears to be the case with sugar. It should be noted that this discussion may not include natural sugars that are found in fruit and dairy. A user may not gain weight or develop chronic diseases from eating too many apples. Rather, this discussion is directed to added sugar (e.g., table sugar, high-fructose corn syrup, honey) that may be found in approximately 74% of packaged foods sold in supermarkets.

The average American consumes about 22 teaspoons (350 calories) of added sugar every day. About half of this added sugar is coming from sweetened beverages, like soda, sport and fruit drinks, teas, and energy drinks. That's about 3 times the amount recommended by the American Heart Association (9 teaspoons for men and 6 teaspoons for women). Sugar intake at this level has been shown to cause increases in weight, triglycerides, blood pressure, and insulin resistance, all of which increases our risk of heart disease and diabetes. While many users understand that added sugar is unhealthy, these users might not realize how much sugar is being consumed in everyday foods.

While added sugar is fine in moderation, but just like any other nutrient (protein, fat, fiber, vitamins, minerals, etc.), too much can become a problem. Taking steps to decrease the sugar intake can help you set up a healthy lifestyle.

The nutrition skill of eat less sugar includes the nutrition subskills of identifying sources of sugar and limiting added sugar intake to less than 30 grams per day. The nutrition skill of identifying sources of sugar may include reading labels. However, it may be difficult to determine natural sugars from added sugars. While newer nutrition labels may list added sugar separately, not all products do this. To determine the amount of added sugar, a user may compare the unsweetened version to the sweetened version. Furthermore, added sugar may be inferred from the ingredient list. Added sugars are often call by names, including: honey, brown sugar, confectioners or powdered sugar, corn syrup, corn syrup solids, dextrose, fructose, sucrose, high-fructose corn syrup (HFCS), invert sugar, malt syrup, maltose, maple syrup, molasses, nectars (like peach nectar, pear nectar, etc.), and pancake syrup.

A user may keep a food journal for a period of time and write down foods that contribute to added sugar each meal/day. This may help the user to become aware of the amount of added sugar consumed, without stressing about reducing the amount of added sugar.

The nutrition subskill of limiting added sugar intake to less than 30 grams per day may include reviewing the food journal prepared in the previous nutrition subskill and choosing less sugary options for meals. This may allow a user to substitute sugar, without completely eliminating it.

### Nutrition Skill 9: Honor Hunger

Many people home from work, start making dinner, and eat crackers, cereal, chocolate chips, and juice all before dinner is made. Then they start eating not really hungry, but since dinner was made, they feel obligated to eat it, since crackers and chocolate chips aren't a “good dinner.” Plus, they need the protein and vegetables. Some people may describe a “lack of willpower” as they mindlessly eat. However, a person may not have a lack of willpower, but may be experiencing a biological response to hunger. When people skip or skimp on a meal, or snack and get overly hungry, they often end up eating excess food as our body reacts to increased hunger and releases chemicals and hormones to encourage eating. Neuropeptide Y, in particular, is released in response to hunger and triggers the desire to eat carbohydrates. Simple carbohydrates are the most tempting, as they are quick fuel. This is a good thing, as the body is trying to keep itself from starving. However, this response is often interpreted as losing control, and blamed on a lack of willpower, rather than based on an understanding of biology.

Oftentimes, people allow themselves to get overly hungry by skipping meals and snacks. They also sometimes eat inadequate calories and set themselves up for overeating. Honoring hunger and fullness may help a user to combat this challenge.

The specific goals for quantities discussed in the previous nutrition skills may provide a user with a good feeling of what it takes to eat healthy. However, a user may not wish to count calories and measure portions indefinitely. After developing healthy habits, including an understanding of how to incorporate fruits, vegetables, whole grains, and protein in a way that works for the user, the user may begin listening to her body. Everyone is unique. Thus, a user may need more or less protein at breakfast to become full or may need more or fewer snacks than another. The user's body is the real expert, and so she should listen to it. The skills developed in the previous nutrition skills can help the user craft a healthy diet. However, these skills are not intended to be rigid rules. A user should not eat an extra snack if she is really hungry. That just sets her up to overeat later. The user should also not eat when she is not hungry just to get every gram of protein or every serving of vegetables recommended. Following a hunger scale can help the user get in touch with her body, be mindful about her eating, and avoid over or under eating.

The nutrition skill of honor hunger includes the nutrition subskills of getting in touch with hunger cues by using a hunger scale, getting in touch with fullness cues and practice not finishing a plate, and coping with emotions without food. The nutrition subskill of getting in touch with hunger cues by using the hunger scale includes recognizing the cues for hunger. These cues might include a growling stomach, irritability, lightheadedness, headaches, fatigue, or difficulty concentrating. It can be hard to recognize these cues when a user is busy, distracted, or consciously ignores them for long periods of time. That's when the user goes from subtle cues to strong biological reactions and overeating. To help avoid this scenario, the user may catch those subtle hunger cues and eat before getting overly hungry. A hunger/fullness scale may be valuable.

FIG. 2 is a representation of a hunger/fullness scale. The hunger/fullness scale is a scale between zero and ten, with zero being ravenously hungry and ten being sick from eating too much. A user should set a goal to start eating at level three or four, which is hungry, but not ravenous. This may help prevent the user from becoming ravenous and overeating in response.

The nutrition subskill of getting in touch with fullness cues and practice not finishing a plate may include identifying fullness cues. A user may have an aversion to wasting food, however, the user should determine if he is eating because he is hungry, or simply because the food is in front of him. Oftentimes, a user may be satisfied with less than the amount provided to him. A user may stop eating during the meal and rate his fullness. When the user reaches a fullness level of a six or a seven, the user should stop eating. This may include throwing away food, which may be preferable to overeating.

The nutrition subskill of coping with emotions without food may help a user to stop eating from emotional hunger. Eating is an emotional experience from the first day of life. People use it to comfort, celebrate, reward, distract, and even as religious symbolism. It is normal to eat emotionally on occasion. For example, a user might choose to eat chicken noodle soup for comfort when sick, even if she is not hungry. Emotional eating becomes problematic when it the only way a person deals with emotions. Food might help distract from stress at work, but it won't fix it. It provides momentary comfort, but won't solve a person's problems.

Thus, a user may identify emotional eating and find ways to cope with her emotions other than food. Before eating, a user may stop and rate her hunger. If you are not biologically hungry, ask she may ask herself, “What am I feeling right now?” She may assess her emotional state as including: stressed, bored, lonely, sad, angry, tired, or do you want to fit in with others who are eating? Next, she may ask herself, “What can I do to help myself instead of eating?” Maybe it is calling a friend, going for a walk, meditating, watching a movie, punching a pillow, or asking for help to resolve a problem. Once the user has identified why she is eating when she is not hungry, may decide how to cope with the emotion without food.

### Nutrition Skill 10: Increase Variety

People often get stuck in a rut when trying to eat healthy. A person may figure out a meal that works well for him, and then eat it over and over and over again. For example, a person may become comfortable with to meals including chicken, brown rice, and broccoli, and they eat it exclusively. There is nothing wrong with this meal, but the lack of variety may cause the user to experience diminishing returns. Foods all have their own unique makeup. The nutrients that make a carrot orange, hard, and smell like a carrot, are different nutrients than what makes kale green, soft, and bitter.

Just like the body needs all of the essential vitamins and minerals, it needs a variety of phytochemicals, too. To help the body thrive, it does not need a lot of a few healthy foods, it needs a large variety of healthy foods. This not only helps the body to get more nutrients, but also may make healthy eating more enjoyable.

Another important note for variety is cooking in a variety of ways. Eating a variety of foods, cooked a variety of ways, including preparing some foods raw, may provide a user with a variety of different nutrients that may be released, expressed, and/or altered based on the preparation method. In this manner, the user may receive the benefit from a multitude of nutrients to prevent disease.

The nutrition skill of increase variety includes the nutrition subskills of eating four or five colors of fruits and vegetables every day, eating protein from a variety of sources, eating a variety of grains, and using different cooking methods. The nutrition subskill of eating four or five colors of fruits and vegetables every day may help a user to receive a variety of vitamins, minerals, and phytochemicals.

The nutrition subskill of eating protein from a variety of sources may include eating protein from vegetables, legumes, nuts, seeds, and grains. This may help a user to receive a variety nutrients. The nutrition subskill of eating a variety of grains may help a user to receive different amounts of fiber, protein, and other nutrients from the grains.

The nutrition subskill of using different cooking methods may change the bioavailability of different nutrients. Therefore, by preparing the same foods in different ways, the nutritional benefit of the food may change. A user may prepare food by roasting, grilling, sautéing, broiling, slow cooking, baking, boiling, simmering, steaming, blanching, poaching, pressure cooking, stewing, other cooking methods, and combinations thereof.

### Nutrition Skill 11: Healthy Dining Out

Many people eat at restaurants, especially while traveling. When traveling, a user may consider planning ahead. For example, the user may preview the menu before attending a restaurant to pre-select a meal. Furthermore, a user may check the nutrition facts of a meal on a menu. The user may set a goal for the same nutrition goals while eating out, such as filling half the plate with vegetables and so forth. The user may need to ask the restaurant for substitutions. Restaurants may provide too much food for a user, and the user may leave food on the plate or order from the children's menu.

The nutrition skill of healthy dining out includes the nutrition subskills of planning ahead and looking for trigger words, making substitutions, and paying attention to how the user feels. The nutrition subskill of planning ahead and looking for trigger words may help a user to avoid making an on-the-spot decision regarding a meal. Furthermore, the user may learn to avoid unhealthy options by spotting trigger words such as loaded, creamed, fried, au gratin, breaded, buttered, buttery, marbled, candied, crusted, smothered, and combinations thereof.

The nutrition subskill of making substitutions may help the user to reach the goal for a specific type of food, such as vegetables or whole grains. This may help the user to maintain healthy habits while eating at a restaurant.

The nutrition subskill of paying attention to how the user feels may include pausing while eating to assess his hunger/fullness. Restaurant portions are often too large, and therefore the user may need to stop eating before the plate is done to assess whether he is still hungry, or eating socially and/or mindlessly simply because there is food on the plate in front of him. Assessing hunger/fullness levels may help the user with portion control.

### Nutrition Skill 12: Meal Planning

Many people spend significant amounts of money eating at restaurants. Eating at restaurants may make it difficult to eat healthy and limit portion sizes. Planning healthy meals may help a user to eat healthy and may save the user money by purchasing fewer restaurant meals in a week.

The nutrition skill of meal planning may include the nutrition subskills of planning dinner, packing lunch, and planning breakfast. The nutrition subskill of planning dinner may include picking meals that the user desires to make, and then making a shopping list to avoid purchasing more than is necessary. This may help encourage healthy eating by limiting purchasing food at the end of the day because no plan is in place.

Planning dinner may include planning based on a schedule that includes dinner with friends, social events, sporting events, and so forth. Furthermore, planning dinner may include planning on leftovers. This may include making more than a single meal's worth of food, and refrigerating and/or freezing the extra food to provide food for future meals.

The nutrition subskill of packing lunch may help save the user may include packing leftovers for lunch, making grain bowls with leftover vegetables, and/or making healthy sandwiches. This may save the user money and improve the health of lunches.

The nutrition subskill of planning breakfast may include pre-packaging smoothies, cooking eggs the night before, or pre-cooking grains or hard-boiled eggs. Because lunch is often rushed, this may help prevent eating a quick, unhealthy breakfast because there is no time to prepare a healthy meal.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for improving a user's health, comprising:
presenting a first nutrition skill to said user, the first nutrition skill including a first nutrition action to be performed by said user;
asking said user a first nutrition-related question based on the first nutrition action after a first elapsed period of time;
receiving a first nutrition progress report based on a first response to the first nutrition-related question;
checking the first nutrition progress report against a first nutrition goal;
if the first nutrition progress report meets or exceeds the first nutrition goal, presenting a second nutrition skill to the user, the second nutrition skill including a second nutrition action to be performed by said user;
asking said user a second nutrition-related question based on the second nutrition action after a second elapsed period of time;
receiving a second nutrition progress report based on a second response to the second nutrition-related question;
checking the second nutrition progress report against a second nutrition goal;
if the first nutrition progress report fails to meet the first nutrition goal, presenting the first nutrition skill to said user again, asking said user the first nutrition-related question after repeating the first elapsed period of time, receiving an updated first nutrition progress report, and checking the updated first nutrition progress report against the first performance criteria.

2. The method of claim 1, further comprising setting a reminder to perform the nutrition action.

3. The method of claim 1, wherein receiving the first nutrition progress report includes said user marking the first nutrition action as complete.

4. A method for improving a user's health, comprising:
presenting a plurality of nutrition skills to a user, the nutrition skills including:
eat more vegetables;
eat protein throughout the day;
stay hydrated;
sweeten with fruit;
power up with whole grains;
eat healthy fats;
fill the day with fiber;
eat less sugar;
honor hunger;
increase variety;
healthy dining out; and
meal planning;
setting nutrition goals for each nutrition skill of the plurality of nutrition skills;
advancing said user successively through each nutrition skill of the plurality of nutrition skills based on whether said user meets or exceeds the nutrition goal for a previous nutrition skill.

5. The method of claim 3, wherein advancing said user include asking said user a plurality of follow up questions, the plurality of follow up questions including determining which factors contributed to said user's success.

6. The method of claim 3, wherein the nutrition skill of eat more vegetables includes:
incorporating two servings of vegetables at lunch; incorporating at least two servings of vegetables at dinner; incorporating a vegetable at breakfast; and incorporating dark, leafy greens to get a total of six servings of vegetables a day.

7. The method of claim 3, wherein the nutrition skill of eat protein throughout the day includes: eating a high protein breakfast; incorporating protein into snacks; limiting intake of red and processed meats; and incorporating plant-based proteins into meals.

8. The method of claim 3, wherein the nutrition skill of stay hydrated includes: drinking a glass of water first thing in the morning; drinking a glass of water before each meal; paying attention to urine color; and limiting sweetened beverages.

9. The method of claim 3, wherein the nutrition skill of sweeten with fruit includes:
incorporating fruit at breakfast and snacking on fruit.

10. The method of claim 3, wherein the nutrition skill of power up with whole grains includes: selecting whole-grain options at grocery stores and restaurants; trying new whole grains; and making at least half of grain products whole grains.

11. The method of claim 3, wherein the nutrition skill of eat healthy fats includes: eating fatty, cold-water fish twice a week; consuming one ounce of nuts or seeds daily; and cooking with healthy fats.

12. The method of claim 3, wherein the nutrition skill of fill the day with fiber includes eating between five and ten grams of fiber at breakfast; incorporating fiber-rich foods as snacks; and incorporating at least two sources of fiber during every meal.

13. The method of claim 3, wherein the nutrition skill of eat less sugar includes: identifying sources of sugar and eating less than 30 grams of added sugar per day.

14. The method of claim 3, wherein the nutrition skill of honor hunger includes: using a hunger/fullness scale; identifying fullness cues and not finishing a plate of food; and coping with emotions without food.

15. The method of claim 3, wherein the nutrition skill of increase variety includes: eating four or five colors of fruits and vegetables per day; acquiring protein from multiple sources; eating a variety of grains; and using different cooking methods.

16. The method of claim 3, wherein the nutrition skill of healthy dining out includes: planning ahead and looking for trigger words; making substitutions; and paying attention to fullness.

17. The method of claim 3, wherein the nutrition skill of meal planning includes: planning dinner; packing lunch; and planning breakfast.
